(19) United States
(12) Patent Application Publication
(10) Pub. No.: US 2007/0092561 A1
(43) Pub. Date: Apr. 26, 2007

(54) Water-Soluble Aspirin Composition

(76) Inventor: Don Milne, Locust Valley, NY (US)

Correspondence Address: CERMAK & KENEALY LLP, 515 E. BRADDOCK RD, SUITE B, ALEXANDRIA, VA, 22314, US

(21) Appl. No.: 11/426,489

(22) Filed: Jun. 26, 2006

Related U.S. Application Data
(60) Provisional application No. 60/693,524, filed on Jun. 24, 2005.

(51) Int. Cl. A61K 31/60; A61K 9/20

(52) U.S. Cl. 424/464; 514/165

(57) ABSTRACT

Compositions in which aspirin is present in combination with alkaline compounds, especially those containing water of crystallization (hydrates), deteriorate on standing. This deterioration may take several forms: It can be a physical deterioration in which such aspirin compositions become completely unmanageable, wet, gummy, sticky masses; or chemical decomposition in which aspirin loses its molecular structure chiefly by losing the acetyl group. The latter is accompanied by formation of acetic acid, the mixture developing its characteristic acetic odor. In both cases, such compositions become entirely unsuitable for all practical commercial and medicinal purposes. Yet, when preparation of water-soluble aspirin compositions is desired, it is impossible to avoid the use of alkaline compounds. This is because the only known method of converting aspirin into soluble form is by means of reacting it with an alkaline compound to form the soluble salt of aspirin. Unexpectedly, it was discovered and is the substance of the present invention that there are two compounds both of which are alkaline and contain water of crystallization (hydrates), and which, in combination with aspirin, give soluble compositions of outstanding stability. Unexpectedly, it was discovered and is also the substance of the present invention that there are several sugar substitutes which have demonstrated an acceleration of absorption into the bloodstream and have demonstrated a reduction in acididity which in turn should reduce or eliminate the potential for bleeding in the stomach. These two compounds are sodium citrate (tri) dihydrate, and potassium citrate (tri) monohydrate. Artificial sweeteners can also be combined in the soluble aspirin composition.

This application claims the benefit of U.S. provisional application 60/693,524, filed Jun. 24, 2005 under 35 U.S.C. §119(e).

### BACKGROUND OF THE INVENTION

The present invention relates in general to aspirin compositions, and in particular to a new and useful water-soluble aspirin composition and the method of making the same.

Aspirin is the most widely used drug in the world. It has a number of important uses in medicine: It is a valuable analgesic, antipyretic, and heart-attack and stroke-preventive. It is one of the most potent anti-inflammatory agents, and is the drug of choice and mainstay of arthritis therapy. It stimulates the immune system, reduces opportunistic infections and is potentially useful as an adjunct in treating cancer, AIDS, and other immune disorders. It shows promise in treatment of Alzheimer's Disease; it is used in rheumatic fever, gout and cataracts; it provides pain relief from tendonitis, headaches, backaches, muscle strains, and other injuries. It has a specific analgesic effect in migraine headaches, a condition in which acetaminophen and ibuprofen show no activity. No other drug in the history of medicine has exhibited such an array of multifaceted therapeutic properties.

Despite all these important medical applications, aspirin is known chiefly for its analgesic properties. Its range of application is greatly reduced by virtue of the fact that aspirin is insoluble (sol: 0.3%). Undissolved aspirin particles adhere to the gastrointestinal mucosa, causing well-known side-effects: gastric irritation, inflammation, heartburn, nausea and pain. Such side-effects occur in about 2-10% of aspirin users. In chronic arthritis, they occur in about 25%. Prolonged contact with aspirin particles produces lesions in the mucosa of the mouth, stomach, rectum and in most other mucosal tissues.

An additional disadvantage of aspirin's low solubility is that millions of drug consumers have swallowing problems and need liquid medication. By some estimates, 20% of all adults are affected, including those suffering from arthritis, Parkinsonism, multiple sclerosis, Lou Gehrig's disease, and others. It is significant that 30% of the popular acetaminophen product, Tylenol (a trademark), had been in capsule form to facilitate swallowing. (Capsules were discontinued for reasons of criminal tampering and contamination in a publicized poisoning incident.)

Because of these disadvantages, aspirin is not widely used as an anti-inflammatory agent, even though it is actually the mainstay and drug of choice in arthritis—a disease directly caused by inflammation. Instead, its use in arthritis is limited mostly to alleviating pain, for which low 325-500 mg dosages suffice. To be an effective anti-inflammatory agent, daily aspirin dosages of 5,000+ mg are required. At such levels, large amounts of undissolved aspirin particles adhere to the gastrointestinal mucosa, greatly aggravating topical irritation and side-effects.

It is thus clear that a water-soluble form of aspirin, free of the undissolved particles which cause the side-effects mentioned, would be highly desirable. Indeed, efforts to produce it date from the discovery of aspirin itself about a hundred years ago.

A number of soluble aspirin salts were developed and used commercially in the past: lithium (“Hydropyrin”), sodium (“Catalgine”), calcium (“Kalmopyrin,” “Ascal,” “Dispril,” “Kalsetal,” “Solaspin,” “Solprin,” “Tylcasin,” “Alcacyl,” “Calurin,” “Ironin,” “Solupsan”) and magnesium (“Novacyl”, the names in the parentheses being trademarks for products each with the particular salt. None of these products proved satisfactory. Some are toxic (lithium salt). Others are contra-indicated in certain conditions such as hypertension (sodium salt), and still others present undesirable pharmacological side-effects (calcium salts are constipating, magnesium salts are laxative). However, the most important disadvantages of these salts were their difficult and expensive manufacture, unpleasant taste, and lack of stability. Of these disadvantages, the most serious was lack of stability. Consequently, none of these products has survived.

Similarly, the disadvantages of commercial products containing aspirin in soluble form available today are these: “Aspro-Clear” and “Upsarin” are high in sodium; while “Aspegic” contains the unnatural d-form of the amino acid lysine, and could not win regulatory approval in the U.S. Other such products, “Disprin” and “Boots” dissolve incompletely and thus do not solve the problem of gastric irritation caused by undissolved aspirin particles. The names in quotes are also trademarks.

In the U.S., the only commercial product containing aspirin in soluble form is “Alka-Seltzer” (another trademark). However, its use as an anti-inflammatory agent, where daily dosages of 5,000 mg and more are required, would mean ingesting some 9,000-10,000 mg of sodium, making the product unacceptable and even dangerous for many consumers.

The simplest and technically the most feasible, economical way to produce aspirin in soluble form would be to formulate aspirin as a simple mixture with alkaline compounds such as bicarbonates, carbonates, acetates and the like. Unfortunately, such formulations rapidly deteriorate, have a short shelf-life, and are thus unsuitable for commercial use.

This deterioration of aspirin is due chiefly to two factors: Presence of water either in a free state such as moisture, or water combined as water of hydration or crystallization; or the presence of alkaline substances.

These facts are disclosed throughout the technical and patent literature on aspirin. For example, the Dispensatory of the United States, 25th ed., p. 16 states:
“. . . Hydrolysis occurs in mixtures of aspirin with hygroscopic substances or salts containing water of hydration.”
Also, The Merck Index, 11th ed., p. 134:
“. . . Powders containing aspirin with an alkali salt such as sodium bicarbonate become gummy in the air. Hydrolysis occurs in admixture with salts containing water of crystallization.”

These well-known facts of aspirin's incompatibility and vulnerability to decomposition by hydrated and alkaline compounds are confirmed and illustrated by the following examples. In all examples, aspirin mesh #80 was used.

The compositions described were all kept at 50.degree.-55.degree. C. This is a generally accepted accelerated aging test. It correctly indicates the probable behavior of such compositions at room temperature. Directly measuring stability at room temperature, while more significant, is impractical, requiring tests of several years' duration.

### REFERENCE EXAMPLE 1

A mixture of 5 g aspirin and 5 g citric acid monohydrate was kept at 50.degree.-55.degree. C. After 40 minutes at this temperature, the mixture became a wet sticky mass and had the odor of acetic acid (the usual decomposition byproduct of aspirin).

### REFERENCE EXAMPLE 2

A mixture of 5 g aspirin and 5 g citric acid anhydrous was kept at 50.degree.-55.degree. for 30 days. In contrast to Example 1 where citric acid monohydrate was used, this mixture, even after 30 days, was unchanged in appearance, and was dry, free-flowing and odorless.

The same deterioration as described in Example 1 was observed with compositions of aspirin with the following hydrates: magnesium chloride hexahydrate, magnesium acetate tetrahydrate, calcium chloride hexahydrate, calcium acetate dihydrate, and sodium phosphate (di) dodecahydrate.

Reference Example 3, below, illustrates the behavior of aspirin in combination with compounds which are both alkaline in nature and contain water of crystallization.

### REFERENCE EXAMPLE 3

The deterioration in Examples 1 and 2 also occurred in aspirin mixtures containing sodium carbonate monohydrate, potassium carbonate sesquihydrate, sodium acetate trihydrate.

Compositions containing aspirin in combination with anhydrous alkaline compounds are equally unstable. Thus, compositions of aspirin with sodium carbonate, potassium carbonate, sodium bicarbonate and potassium bicarbonate all decompose within several hours at 50.degree.-55.degree. C.

The foregoing data show the high instability of compositions containing aspirin in combination with compounds containing water of crystallization (hydrates), or with compounds of alkaline nature, or with compounds that are both of alkaline nature and contain water of crystallization (hydrates).

### SUMMARY OF THE INVENTION

The substance of this invention is the discovery of two compounds, which are both alkaline in nature and contain water of hydration and which, in contrast to all other such compounds, may be combined with aspirin to form compositions that are stable for all medicinal, practical, and commercial purposes. It was unexpected and surprising to discover that sodium citrate (tri) dihydrate and potassium citrate (tri) monohydrate are two compounds which in combination with aspirin exhibit both solubility in water and outstanding stability.

In another embodiment of the invention, these compounds can be mixed with sugar or an artificial sweetener to render them more palatable, demonstrating an acceleration in absorbtion into the bloodstream which may partially take place in the mouth, and may reduce or eliminate potential bleeding in the stomach and gastrointestinal track and have no detrimental effects to the stability of the product. Standard sugar compositions can be used. Any artificial sweetener which is known can be used in the present invention, and is generally incorporated into the composition in smaller amounts proportionally than sugar. The actual amount of artificial sweetener added to the soluble aspirin product to provide an isosweetness to sucrose will vary depending on the sweetness strength of said artificial sweetener. One skill in the art of artificial sweeteners is aware how much of an artificial sweetener is equivalent to a corresponding sweetening amount of sucrose.

The artificial sweeteners contemplated for formulation into the soluble aspirin product include any known artificial sweetener, for example, L-aspartyl-L-phenylalanine methyl ester, 3-(L-aspartyl-D-alanamido)-2,2,4,4-tetramethylthietane (U.S. Pat. No. 4,411,925), saccharin and base salts thereof, potassium 6-methyl-1,2,3-oxathiazin-4(3H)-one-2,2-dioxide (U.S. Pat. No. 3,689,486) and cyclamate and base salts thereof, xylitol-based products, Manno-Max D-Mannose, Maltitol, and sucralose-based products, dipotassium glycyrrhizinate, stevia, somatin, cyclamates, acesulfame K, and thaumatin.

Water-soluble artificial sweeteners such as the soluble saccharin salts, i.e., sodium or calcium saccharin salts, cyclamate salts, acesulfame-K and the like, and the free acid form of saccharin, dipeptide based sweeteners such as L-aspartyl-L-phenylalanine methyl ester and material described in U.S. Pat. No. 3,492,131 and the like are also encompassed as the artificial sweetener of the present invention. Brand name products such as Splenda, SmartSweet, Sweet & Low, Equal, and the like, are also contemplated as the artificial sweetener component of the present invention.

Sucralose (marketed under the tradename SPLENDA.RTM.) is a compound having the CAS Registry Number: 56038-13-2 and the CAS Index Name: 1,6-dideoxy-b-D-fructofuranosyl-4-chloro-4-deoxy-.alpha.-D-galactopyranosi de and is characterized as an intensely sweet, trichlorinated carbohydrate, structurally similar to sucrose, having approximately 600 times the sweetening power of sucrose.

Mixtures of artificial sweeteners, such as a ratio of 10 parts cyclamate to 1 part saccharin, have also been found to have synergistic sweetening properties and improve taste characteristics.

There are specific types of dryers which can be employed for the removal of hydration from the combined composition. These dryers include, but are not limited to, a stainless steel rotary cone dryer (5 ft³ working capacity), a stainless steel vacuum oven (7.5 ft³), and a stainless steel stokes vacuum tumble dryer. In addition, the de-hydratyed composition is further dryed and separated by a Stokes Tornado Mill.

The present invention also encompasses the use of Fluid bed technology & dryer for the final drying and processing of the soluble aspirin composition. Such technology includes, but is not limited to, the ACT 100N Lab Fluid Bed Granulator and Dryer 5 liter bowl capacity, typical charge weight 0.2-3 kg Full instrumentation Variable speed Blower: 70 cfm@40″wc Maximum Air temperature 125 C using a 3 kw heater 220V single phase, 60 hz. Air Atomizing Spray System using 4 bar (60 psi) compressed air for top or bottom spray Touch operated pulsed air filter cleaning.

Accordingly, an object of the present invention is to provide an aspirin composition which is highly soluble in water yet has outstanding stability and shelf life, as has demonstrated a unique ability in absorption into the body and demonstrates a reduction in bleeding in the stomach and gastrointestinal track. The pH of the composition is much less acidic and should be less harmful to the gastric lining of the stomach.

A further object of the present invention is to provide a method for making such a composition.

The various features of novelty which characterize the invention are pointed out with particularity in the claims annexed to and forming a part of this disclosure. For a better understanding of the invention, its operating advantages, and specific objects obtained by its uses, reference is made to the following description of preferred embodiments of the invention.

### DESCRIPTION OF THE PREFERRED EMBODIMENTS

The following are several examples of various compositions and methods of the present invention:

### EXAMPLE 4

Twenty grams of aspirin were thoroughly mixed with 80 g potassium citrate (tri) monohydrate. The mixture is odorless and free-flowing. It was kept for 30 days at 50.degree.-55.degree. and remained free-flowing and odorless: no decomposition was detected.

### EXAMPLE 5

In this example, 80 g of sodium citrate (tri) dihydrate was used and the same degree of stability was observed.

The high degree of stability of the compositions of Examples 4 and 5 makes them suitable for commercial medicinal applications.

These compositions are readily soluble in water and resulting solutions are complete, clear and palatable. It is emphasized that the completeness of these solutions is crucial since, as mentioned before, it is undissolved aspirin particles which adhere to gastrointestinal mucosa causing aspirin's well-known side-effects (heartburn, irritation, nausea and pain).

When preparing solutions of the compositions of this invention, it is advantageous to add a small amount of surface-active agent.

For example, 0.1 to 0.5% weight of surface-active agent can be used with 10% weight aspirin, 20% to 40% weight hydrated salt, all in about 150 ml water.

### EXAMPLE 6

Twenty grams of aspirin, 80 g potassium citrate (tri) monohydrate, and 20 mg of sodium lauryl sulfate (commercially available under the trademark “Empicol”) are thoroughly mixed together. In order to administer a 500-mg dose of aspirin, 2.5 g of this composition are dissolved in 150 ml water. The solution is complete, clear and palatable.

### EXAMPLE 7

Twenty grams of aspirin, 80 g sodium citrate (tri) dihydrate, and 20 mg of “Empicol” are thoroughly mixed together. In order to administer a 500-mg dose of aspirin, 2.5 g of this composition are dissolved in 150 ml water. The solution is complete, clear and palatable.

While compositions containing sodium citrate (tri) dihydrate and potassium citrate (tri) monohydrate are both hydrated salts (tri) of citric acid, and suitable for aspirin therapy, the composition of Example 6 has the advantage of being sodium-free. Since arthritis is a disease afflicting the elderly in particular, and because they are often on sodium-restricted diets, this composition may be preferred.

### EXAMPLE 8

30 g of aspirin, 70 g of Potassium citrate, 100 g of Sugar, and 0.6 g of Empicol (10%) were mixed as detailed below. The aspirin content of this mixture is 15%, therefore 3.3 g equals 500 mg aspirin and 6.6 g equals 1,000 mg aspirin.

These ingredients are thoroughly mixed in a 500-ml Erlenmeyer. The test sample contained 6.6 g (1,000 mg aspirin) (ca. ¾-tasblespoonful or heaping teaspoon). It quickly and neatly dissolves in 150 ml of room-temperature water. The taste is slightly sweet.

The aspirin used is mesh #80. It is first screened on sieve mesh #80 to remove larger particles, about 5%.

Potassium citrate used in this preparation is heat-treated as follows: 200 g passed through mesh #20 is heated on an aluminum plate with frequent stirring at 110-115° for 2 to 2½ hrs. During the heating there is formation of crusts on the surface, and these are broken up from time to time with a flat spatula or tablespoon. The product is placed in a pre-heated 500-ml Erlenmeyer and stoppered with a rubber stopper.

Sugar used in this preparation is granular, and is heated in the same manner as the potassium citrate. Crust formation has been observed during heating indicating the presence of some water.

Empicol 10% is 10 g Empicol and 90 g sugar.

The resulting soluble aspirin (LIQUIPRIN) is hygroscopic at relative humidities of above 60%. Accordingly, its manufacture, handling and packaging should be done below RH 60%. Packaging must be moisture-proof (e.g., airtight envelopes).

The stability characteristics of the final product are as follows: Kept at 50° for 30 days, or 37° for 90 days, the product remains odorless, free-flowing, and free of non-aspirin salicylates.

### EXAMPLE 9

30 g aspirin, 70 g potassium citrate, 100 g sugar, 0.6 Empicol (10%) were mixed as detailed below. The aspirin content of this mixture is 15%, therefore 3.3 g of final product contains 500 mg aspirin and 6.6 g of final product contains 1,000 mg aspirin.

These ingredients are thoroughly mixed in a 500-ml Erlenmyer, then spread on an aluminum plate (11 in.×9×1¼), and placed in the oven and heated 25-30 hours at 55-57° with frequent stirring. Then the mixture is placed in a preheated 500-ml Erlenmeyer and stoppered. The test sample contained 6.6 g (1,000 mg aspirin) (ca. ¾-tablespoonful or heaping tablespoon). It quickly and neatly dissolves in 150 ml of room temperature water. The taste is slightly sweet.

Aspirin mesh #100 is preferred; however, mesh #80 may also be used, but should first be screened on screen #80 to remove larger particle, about 5%. Mesh #80 is preferable because it's commercially available. Meshes #100 and #200 are not commercially available.

The potassium citrate used in this preparation is heat-treated as follows: 200 g passed through mesh #20 is heated on an aluminum plate with frequent stirring at 110-115° for 2 to 2½ hrs. During the heating there is formation of crusts on the surface, and these are broken up from time to time with a flat spatula or tablespoon. The product is placed in a pre-heated 500-ml Erlenmeyer and stoppered with a rubber stopper.

Sugar used in this preparation is granular, and is heated in the same manner as potassium citrate. Crust formation has been observed during heating indicating the presence of some water.

Empicol 10% of 10 g Empicol and 90 g sugar.

The resulting soluble aspirin (LIQUIPRIN) is hygroscopic at relative humidities of about 60%. Accordingly, its manufacture, handling and packaging should be done below RH 60%. Packaging must be moisture-proof (e.g., airtight envelopes).

The stability characteristics of the product are as follows: Kept at 50° for 30 days, or 37° for 90 days, the product remains odorless, free-flowing, and free of non-aspiring salicylates.

### EXAMPLE 10

Analysis for Non-Aspirin Salicylates in soluble aspirin product (LIQUIPRIN):

An amount of LIQUIPRIN containing 100 mg of aspirin (1,000 mg for 10% formulation) is stirred with 10 ml iso-propanol in a wide test-tube.

The insoluble potassium citrate is filtered.

The filtrate must be clear. Five milliliters of filtrate (which now contains 50 mg of aspirin) is transferred to a Nessler Tube.

Water is added to the upper mark and the rube swirled to obtain a homogenous solution.

Four drops of acetic acid and 8 drops of the alum solution are added.

The color is compared with that of a standard salicylic acid solution.

(See the Coplan's Directions [Page ‘B’] for further details).

NOTE: Usually no color develops with LIQUIPRIN, indicating absence of non-aspirin salicylates.

If no color develops, add a drop of salicylic acid solution to ensure that no false negative is present, due perhaps to a slight amount of potassium citrate that might have slipped through. These drops of salicylic acid must produce color.

The % non-aspirin salicylates, if present, indicates the degree of decomposition of aspirin in LIQUIPRIN. Small amount of non-aspirin salicylates (less than 3%) are permissible in a buffered aspirin-formulation.

Test for Salicylate

(Coplans)

1. Reagents
a) Ferric Solution: 1 g Fe Alum in 200 ml water containing 1 ml HCl conc.
b) Standard Salicylic Solution: 1 gm in 1,000 ml water (0.1%)

2. Color Ccomparison Test-Tubes (Nessler)
Length: 154 mm; ID: 19 mm; OD: 22 mm

3. Procedure:

dissolve 50 mg of compound in 20 ml of water, place into Nessler, add 4 drops of glacial acetic acid, followed by 8 drops of ferric solution

place 20 ml of water in the second Nessler, add 4 drops of glacial acetic acid, add 8 drops of ferric solution; then, with shaking, add dropwise the 0.1% salicylic solution until the color matches that of the solutions in the first Nessler (viewing through the width, no eh depth, of the Nessler, and using a white background.)

Each drop of 0.1% salicylic solution corresponds to 0.05 mg of salicylic acid.

The concentration of the solution being tested must be so adjusted that not more than 6-7 drops of salicylic solution is required; otherwise the color is too deep for comparison.

4. Note:

The test requires less than 5 minutes to perform. Since aspirin salts hydrolyze in water, the test must be completed as fast as possible.

### EXAMPLE 11

30 g of aspirin, 70 g of Potassium citrate, 60 g of artificial sweetener, and 0.6 g of Empicol (10%) can be mixed as detailed below. The aspirin content of this mixture is 37.5%, therefore 4.4 g equals 1650 mg aspirin.

These ingredients are thoroughly mixed in a 500-ml Erlenmeyer. The test sample can contain 4.4 g (1,650 mg aspirin) (ca. ¾-tasblespoonful or heaping teaspoon). It quickly and neatly dissolves in 150 ml of room-temperature water. The taste is slightly sweet.

The aspirin used is mesh #80. It is first screened on sieve mesh #80 to remove larger particles, about 5%.

Potassium citrate used in this preparation is heat-treated as follows: 200 g passed through mesh #20 is heated on an aluminum plate with frequent stirring at 110-115° for 2 to 2½ hrs. During the heating there is formation of crusts on the surface, and these are broken up from time to time with a flat spatula or tablespoon. The product is placed in a pre-heated 500-ml Erlenmeyer and stoppered with a rubber stopper.

The artificial sweetener used in this preparation may be granular, and is heated in the same manner as the potassium citrate. Crust formation may be observed during heating indicating the presence of some water.

Empicol 10% is 10 g Empicol and 90 g sugar.

The resulting soluble aspirin (SOLUPRIN) is hygroscopic at relative humidities of above 60%. Accordingly, its manufacture, handling and packaging should be done below RH 60%. Packaging must be moisture-proof (e.g., airtight envelopes).

The stability characteristics of the final product are as follows: Kept at 50° for 30 days, or 37° for 90 days, the product remains odorless, free-flowing, and free of non-aspirin salicylates.

While specific embodiments of the invention have been shown and described in detail to illustrate the application of the principles of the invention, it will be understood that the invention may be embodied otherwise, without departing from such principles.

What is claimed is:

1. A water-soluble, stable composition comprising:
aspirin; hydrated salts (tri) of citric acid with metals of the alkaline group, and an artificial sweetener.

2. The composition of claim 1 wherein the alkaline group consists of sodium and potassium.

3. The composition of claim 2 wherein the hydrated salts (tri) of citric acid are selected from the group consisting of potassium citrate (tri) monohydrate and sodium citrate (tri) dihydrate.

4. The composition according to claim 3 containing about 10% by weight aspirin and from about 20% to about 40% by weight hydrated salt, in about 150 ml water.

5. The composition of claim 3 comprising a water solution containing about 10% by weight aspirin, from about 20% to about 40% by weight hydrated salt, in about 150 ml water.

6. The composition of claim 5 including from 0.1% to 0.5% by weight surface-active agent.

7. The composition of claim 6 wherein the surface-active agent is sodium lauryl sulfate.

8. The composition of claim 1 wherein the hydrated salt comprises sodium citrate (tri) dihydrate.

9. The composition of claim 1 wherein the hydrated salt comprises potassium citrate (tri) monohydrate.

10. The composition of claim 1 comprising a solution of acid with dehydrated salt and water.

11. The composition of claim 10 including a surface-active agent in the solution.

12. The composition of claim 1 consisting essentially of about 10% by weight aspirin and from about 20% to 40% by weight hydrated salts, all in water, the hydrated salts selected from the group consisting of sodium citrate (tri) dihydrate and potassium citrate (tri) monohydrate.

13. The composition of claim 1 including about 150 ml water.

14. A method of making a water-soluble and stable composition containing aspirin, comprising:
Combining aspirin with a hydrated salt (tri) of citric acid with metals of the alkaline group and an artificial sweetener.

15. A method according to claim 14 wherein the hydrated salts are selected from the group consisting of sodium citrate (tri) dihydrate and potassium citrate (tri) monohydrate.

16. A method according to claim 15 comprising dissolving the mixture of aspirin and hydrated salts in water.

17. The method according to claim 16 including combining in the solution a surface-active agent.

18. A method according to claim 14 comprising dissolving the aspirin with hydrated salts in water.

19. A method according to claim 18 including utilizing from about 10% by weight aspirin with about 20% to about 40% by weight hydrated salts in water.

20. A method according to claim 19 including dissolving the combination of aspirin and hydrated salts in water and adding from 0.1% to 0.5% by weight surface-active agent.
